(19) United States
(12) Patent Application Publication
(10) Pub. No.: US 2010/0198261 A1
(43) Pub. Date: Aug. 5, 2010

(54) ADJUSTABLE SPINAL STABILIZATION SYSTEMS

(75) Inventors: Hai H. Trieu, Cordova, TN (US); Dimitri K. Protopsaltis, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/757,123

(22) Filed: Apr. 9, 2010

Related U.S. Application Data
(63) Continuation of application No. 11/869,355, filed on Oct. 9, 2007.

(51) Int. Cl. A61B 17/70; A61B 17/88

(52) U.S. Cl. 606/264; 606/279

(57) ABSTRACT

A spinal stabilization system includes a stabilization member with opposite end portions lying along a longitudinal axis and an adjustment mechanism between the end portions that allows the end portions to be moved toward and away from one another along the longitudinal axis.

### BACKGROUND

The spine is subject to various pathologies that compromise its load bearing and support capabilities. Such pathologies of the spine include, for example, degenerative diseases, the effects of tumors and, of course, fractures and dislocations attributable to physical trauma. In the treatment of diseases, malformations or injuries affecting spinal motion segments (which include two or more adjacent vertebrae and the disc tissue or disc space therebetween), and especially those affecting disc tissue, it has long been known to remove some or all of a degenerated, ruptured or otherwise failing disc. It is also known that artificial discs, fusion implants, or other interbody devices can be placed into the disc space after disc material removal. External stabilization of spinal segments alone or in combination with interbody devices also provides advantages. Elongated rigid plates, rods and other external stabilization devices have been helpful in the stabilization and fixation of a spinal motion segment, in correcting abnormal curvatures and alignments of the spinal column, and for treatment of other conditions.

While external stabilization systems have been employed along the vertebrae, the geometric and dimensional features of these systems and patient anatomy constrain the surgeon during surgery and prevent optimal placement and attachment along the spinal column. For example, elongated, one-piece spinal rods can be difficult to maneuver into position along the spinal column, and also provide the surgeon with only limited options in sizing and selection of the rod system to be placed during surgery. Furthermore, there remains a need to provide spinal stabilization systems which correct one or more targeted spinal deformities while also preserving the ability to adjust the systems for optimal fit during the surgical procedure and in subsequent surgical procedures.

### SUMMARY

A spinal stabilization system includes a stabilization member with opposite end portions lying along a longitudinal axis and an adjustment mechanism between the end portions that allows the end portions to be moved toward and away from one another along the longitudinal axis to adjust the length of the stabilization member.

According to one aspect, a spinal stabilization system comprises a stabilization member extending along a longitudinal axis between a first end portion and a second end portion. The stabilization member also includes an adjustment mechanism connecting the first and second end portions along the longitudinal axis. The system also comprises first and second anchor members each including a bone engaging portion to engage a bony structure and a receiving portion extending from the bone engaging portion. The receiving portion is configured to receive a respective one of the first and second end portions. The first and second anchor members further each include an engaging member to fixedly secure the respective end portion to the receiving portion in the receptacle. The adjustment mechanism is operable to move the first and second end portions toward and away from one another along the longitudinal axis to shorten and lengthen the stabilization member along the longitudinal axis with the end portions fixedly secured to the first and second anchor members.

According to a further aspect, a spinal stabilization system comprises a stabilization member extending along a longitudinal axis between a first end portion and an opposite second end portion and an adjustment mechanism connecting the first and second end portions. The adjustment mechanism includes a housing including a sleeve portion defining a bore extending along the longitudinal axis and a mounting portion adjacent to the sleeve portion. The adjustment mechanism also includes an adjustment member extending through the bore between opposite first and second engaging end that are engaged to respective ones of the first and second end portions. The adjustment mechanism also includes a drive member in the mounting portion engaged to the adjustment member. The drive member is operable to manipulate the adjustment member to move the first and second end portions toward and away from one another along the longitudinal axis.

According to another aspect, a method for spinal stabilization comprises: engaging a first anchor to a first vertebra; engaging a second anchor to a second vertebra; engaging first and second end portions of a stabilization member to respective ones of the first and second anchors, the stabilization member including an adjustment member extending between and engaged to the first and second end portions; manipulating the adjustment mechanism to adjust a length of the stabilization member between the first and second end portions while the stabilization member is engaged to the first and second anchors; maintaining the stabilization member in the adjusted length; and manipulating the adjustment mechanism to adjust the adjusted length after maintaining the adjusted length for a period of time.

According to another aspect, a spinal stabilization system comprises a stabilization member extending along a longitudinal axis between a first end portion and an opposite second end portion. The stabilization member includes a length between the first and second end portions sized to extend between at least two vertebrae of a spinal column. The stabilization member includes an adjustment mechanism connecting the first and second end portions. The adjustment mechanism comprises a housing, a first adjustment member extending from the first end portion and to the housing along a first side of the longitudinal axis, a second adjustment member extending from the second end portion and to the housing along a second side of the longitudinal axis, and a drive member engaged to the housing between the first and second adjustment members. The drive member is engaged to the first and second adjustment members and is operable to manipulate the adjustment members to move the first and second end portions toward and away from one another along the longitudinal axis.

Related features, aspects, embodiments, objects and advantages will be apparent from the following description.

### BRIEF DESCRIPTION OF THE DRAWINGS

FIG. 1 is a diagrammatic elevation view of a posterior portion of the spinal column with a stabilization system shown diagrammatically in attachment with the spinal column.

FIG. 2 is an elevation view of a stabilization member in a first, reduced length configuration and anchor members engageable to the stabilization member.

FIG. 3 is the stabilization member of FIG. 2 adjusted to increase the length thereof and with the anchor member engagement locations along the stabilization member shown diagrammatically.

FIG. 4 is the stabilization member of FIG. 2 in another adjusted configuration to increase the length thereof.

FIG. 5 is an elevation view in partial section of the stabilization member of FIG. 4.

FIG. 6 is a cross-sectional view of the stabilization member of FIG. 5 along line 6-6 of FIG. 5.

FIG. 6A is a cross-sectional view of another embodiment stabilization member taken along a location thereof corresponding to the location of line 6-6 of the stabilization member of FIG. 5.

FIG. 7 is an elevation view of another embodiment stabilization member.

FIG. 8 is an elevation view of another embodiment stabilization member.

FIG. 9 is a perspective view of another embodiment stabilization member in a reduced length configuration and a diagrammatic view of one embodiment adjustment device.

FIG. 10 is a perspective view of the stabilization member of FIG. 9 adjusted to increase the length thereof.

FIG. 11 is a cross-sectional view of the stabilization member of FIG. 10 along line 11-11 of FIG. 10.

FIG. 12 is a perspective view of another embodiment stabilization member.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

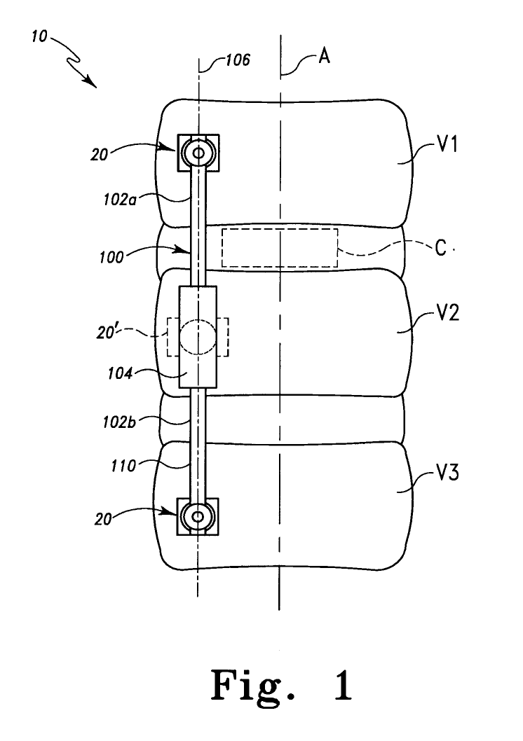

FIG. 1 illustrates posterior spinal stabilization system 10 located along a spinal column of a patient. More specifically, stabilization system 10 can be affixed to vertebrae V1, V2, V3 of the spinal column segment from a posterior approach. Applications along two vertebrae or four or more vertebrae are also contemplated. Stabilization system 10 generally includes one or more anchor members 20 (shown diagrammatically in FIG. 1 and discussed further below) and at least one elongated stabilization member 100 extending generally along central spinal column axis A with a length sized to extend between anchor members 20.

Stabilization member 100 includes an elongated body 110 that extends along longitudinal axis 106. Body 110 of stabilization member 100 includes opposite end portions 102a, 102b extending along longitudinal axis 106 and an adjustment mechanism 104 between end portions 102a, 102b. Adjustment mechanism 104 is operable to selectively move end portions 102a, 102b toward or away from one another along longitudinal axis 106 to increase or decrease the length of body 110 of stabilization member 100. The ability to adjust the length of stabilization member 100 along longitudinal axis is desirable for many applications in spinal surgical procedure. including, but not limited to, applying distraction or compression forces to one or more vertebrae through anchor members 10, applying corrective forces to provide a desired alignment of one or more vertebrae, accommodating growth or other changes in the anatomy of the patient over time, facilitating revision surgery in minimally invasive surgical approaches without replacement of the stabilization member, and maintaining the position or orientation of one or more vertebrae during the implantation procedure and over time. Spinal stabilization system 10 may be used for, but is not limited to, treatment of degenerative spondylolisthesis, fracture, dislocation, scoliosis, kyphosis, spinal tumor, herniation, stenosis, and/or a failed previous fusion.

In one embodiment, adjustment mechanism is located along one of the vertebrae, such as vertebra V2, and is unconstrained relative to vertebra V2. In another embodiment, adjustment mechanism 104 is located along a spinal disc space or other structure between adjacent vertebrae of single level or multiple level procedures. In yet another embodiment, adjustment mechanism 104 is constrained or fixed relative to vertebra V2 with an anchor member 20′, as shown in FIG. 1. In another embodiment, adjustment mechanism 104 is constrained or fixed relative to one of the adjacent vertebrae of a single level procedure. In still another embodiment, adjustment mechanism 104 is semi-constrained so that translation and/or rotation is permitted in or more degrees of freedom relative to the adjacent vertebral structure.

Stabilization member 100 is provided in various embodiments made from any one of a number of materials and stiffness profiles along its length. Stabilization member 100 is provided in one embodiment with a profile that is completely rigid along its length so that minimal or no bending or flexing is provided in response to spinal loading and motion. Such rigid embodiments can be employed in conjunction with spinal fusion of one or more of the vertebrae with one or more spinal implants, bone growth material or other fusion construct, represented as construct C in FIG. 1, between vertebrae of one or more levels of the spinal column. In another embodiment, stabilization member 100 is provided with one or more components that permit limited bending and/or flexing in response to loading and motion from the spinal column for dynamic stabilization procedures. In another embodiment, stabilization member 100 is substantially non-resistant to compression loading and collapsible so that little or no resistance is provided to movement of the vertebrae toward one another along the stabilization member, while the stabilization member provides tensile resistance in response to movement of vertebrae away from one another along the stabilization member 100.

One or more components of stabilization member 100 can be provided with any suitable biocompatible material. Examples of suitable material include titanium and titanium alloys, stainless steel, and other suitable metals and metal alloys; polymers such as polyetheretherketone (PEEK); composites such as carbon-PEEK or titanium-PEEK composites; and any combination of these materials. The end portions of stabilization member 100 are configured to be anchored to bony structure along the spinal column, such as the pedicles, spinous processes, or other posterior elements. Anchoring of stabilization member 100 along the anterior portions of the vertebral bodies is also contemplated, including along the lateral, antero-lateral, and anterior sides of the anterior vertebral body structure.

Illustrative embodiments disclosed herein include spinal stabilization members with end portions in axially aligned relationships. Other embodiments contemplate axially offset relationships, and stabilization members that define one or more curved or arced segments along its longitudinal axis. The stabilization members are engaged to respective ones of first and second vertebrae with an anchor member, while the adjustment mechanism adjustably connects end portions of the stabilization member to one another between the anchor members. The adjustment mechanism permits the length of the stabilization member between the anchor members to be readily increased or decreased either by manual manipulation of the adjustment mechanism; minimally invasive access to the adjustment mechanism, by remote operation of the adjustment mechanism, or by pre-programmed operation or control of the adjustment mechanism.

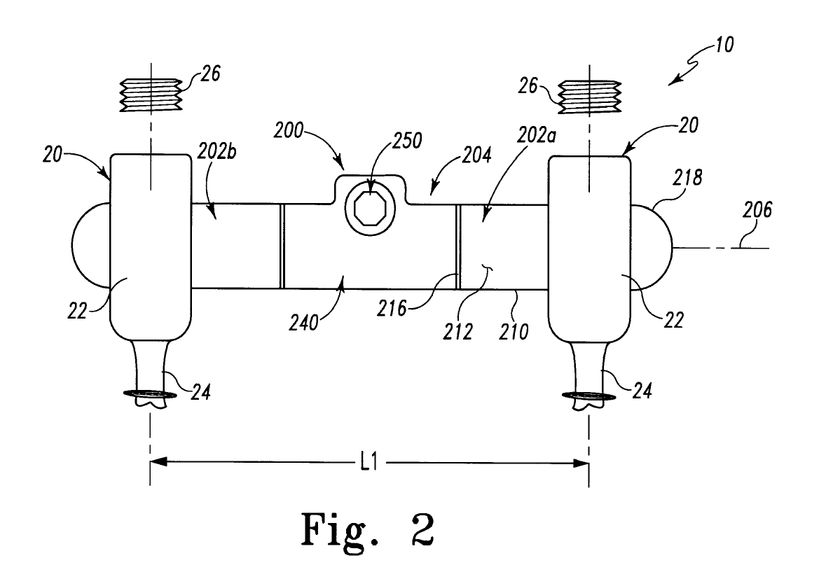

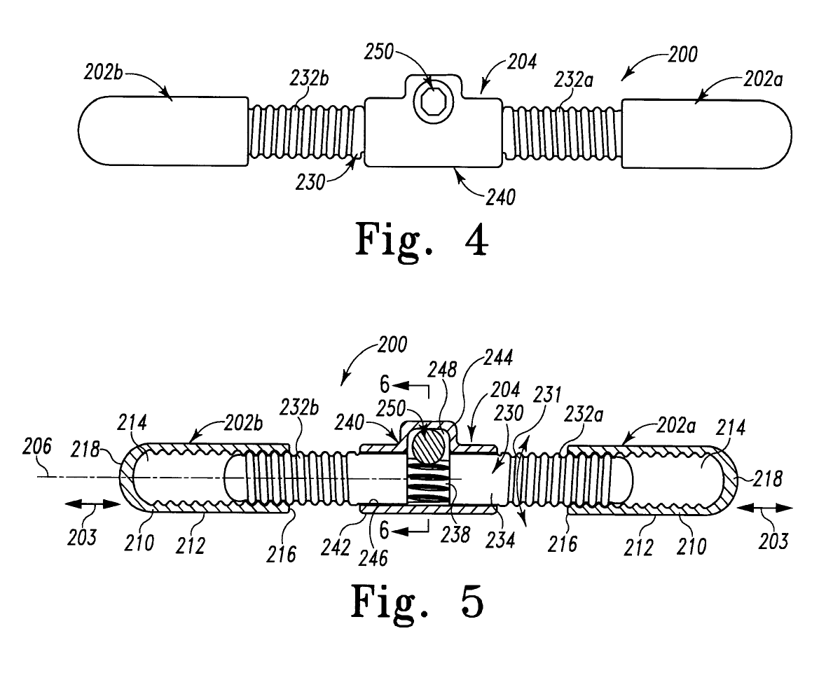

Referring now to FIGS. 2-6, an embodiment of stabilization member 100 will be described with reference to stabilization member 200. Stabilization member 200 includes opposite end portions 202a, 202b extending along longitudinal axis 206 and an adjustment mechanism 204 axially connecting end portions 202a, 202b. End portions 202a, 202b are engageable to bony structure of the spinal column with respective ones of the anchor members 20 in the manner discussed above with respect to stabilization member 100.

In the illustrated embodiment, anchor members 20 include a configuration having a proximal receiver portion 22 and a distal bone engaging portion 24. Bone engaging portion 24 is shown with a threaded shaft in the form of a bone screw. Other embodiments contemplate other forms for bone engaging portions 24, including hooks, staples, rivets, tacks, pins, intrabody devices, interbody devices, cross-link members, clamps, wires, tethers, cables, rods, plates, or any other bone engaging device. Receiver portion 22 can be fixed relative to bone engaging portion 22, or can be movable to provide adjustment capabilities for the receiver portion when the bone engaging portion is engaged to the bony structure. Receiver portion 22 provides a structure for engagement with the respective end portion of stabilization member 200. Some examples of suitable receiver portions include U-shaped saddles, top-loading saddles, side-loading saddles, bottom-loading saddles, and end-loading saddles. The saddles include a receptacle in which the end portion is positioned. Other examples of suitable receiver portions include posts about which the end portion is positioned, a clamp that clamps the end portion to a post or bone engaging portion of the anchor member, or any other suitable engagement structure. In one embodiment, anchor member 20 is a multi-axial screw, and in another embodiment anchor member 20 is a uni-axial screw. Engaging members 26 are provided that engage receiver portion 22 and secure the respective end portion 202a, 202b thereto. In the illustrated embodiment, engaging members 26 are set screws that include external thread profiles to engage internal threads of the respective receiver portion 22. Other embodiments contemplate engaging members 26 in the form of nuts, caps, slide-locking members, washers, snap fit members, interference members, cerclages, clamps, and combinations thereof. In still other embodiments, the stabilization member is engaged to the anchor member without an engaging member.

End portions 202a, 202b are configured identically to one another in the illustrated embodiment, although embodiments with end portions having different configurations are also contemplated. End portions 202a, 202b include a tubular member 210 with a wall 212 extending around a central bore 214. Central bore 214 is open at the inner end 216 of member 210, and is enclosed at the opposite end by outer end wall 218. Other embodiment contemplate that the outer end is open. Wall 212 includes an inner surface 220 extending around bore 214 that defines an internal thread profile along bore 214.

Adjustment mechanism 204 includes an adjustment member 230 extending along longitudinal axis 206 between end portions 202a, 202b. Adjustment member 230 includes opposite engaging ends 232a, 232b that are received in bore 214 of the end portions 202a, 202b, respectively. Engaging ends 232a, 232b each include an external thread profile that threadingly engages the internal thread profile along bore 214 of the respective end portion 202a, 202b.

Adjustment member 230 includes an intermediate portion 234 between engaging ends 232a, 232b. Intermediate portion 234 extends through a housing 240 of adjustment mechanism 204. Housing 240 includes an outer sleeve portion 242 defining a longitudinal bore 246 through which intermediate portion 234 extends, and a mounting portion 244 adjacent to sleeve portion 242. Mounting portion 244 includes a chamber 248 housing a drive member 250 adjacent to and in engagement with intermediate portion 234 of adjustment member 230. Drive member 250 is operable to rotate adjustment member 230 about longitudinal axis 206 in sleeve portion 242. As adjustment member 230 rotates about longitudinal axis 206, end portions 202a, 202b are maintained in rotational position about longitudinal axis 206 by engagement with the respective anchor member 20. The axial rotation of adjustment member 230 rotates threaded engaging ends 232a, 232b along the thread profile of end portions 202a, 202b, causing the end portions 202a, 202b to move toward or away from one another along longitudinal axis 206 and the respective engaging end 232a, 232b, depending on the direction of axial rotation of adjustment member 230.

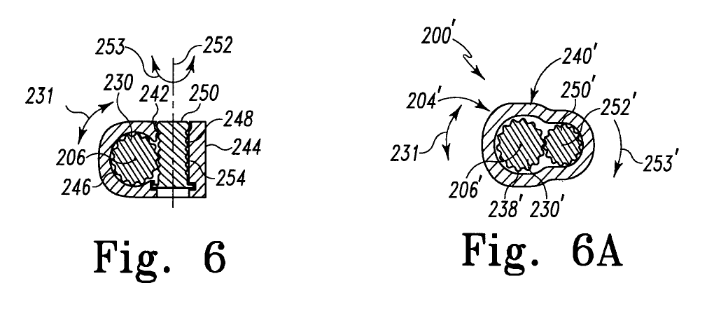

In one embodiment shown in FIG. 6, drive member 250 includes an outer profile 254 that engages a drive structure 238 around the periphery of adjustment member 230. Rotation of drive member about its central axis 252 causes the outer profile 254 to push against the respective adjacent portion of drive structure 238, resulting in adjustment member 230 rotating about longitudinal axis 206. In the illustrated embodiment, drive structure 238 includes a series of spirally oriented teeth spaced circumferentially around intermediate portion 234 so that drive member 250 remains engaged thereto by a thread defining outer profile 254 of drive member 250. The engagement between drive member 250 and drive structure 238 prevents or resists axial rotation of adjustment member 230 unless it is actively rotated by rotation of drive member 250.

In one embodiment, drive member 250 and adjustment member 230 engage one another in a worm-gear type arrangement. In this type of arrangement, drive structure 238 provides a worm gear type of configuration in engagement with teeth or threads about the outer profile 254 of drive member 250. The positioning of drive member 250 and adjustment member 230 relative to one another in this arrangement is infinitely variable to provide infinite number of lengths for stabilization member 200 along longitudinal axis 206.

Drive member 250 extends along and is rotated about its central axis 252, which is transversely oriented to longitudinal axis 206. Rotation of drive member 250 about axis 252, as indicated by arrow 253, causes rotation of adjustment member 230 about longitudinal axis 206, as indicated by arrow 231, which in turn lengthens or shortens stabilization member 200 along longitudinal axis 206 by displacing end portions 202a, 202b away or toward one another, as indicated by arrows 203. Accordingly, axial expansion and retraction of the length of stabilization member 200 is accomplished by manipulating drive member 250 along an axis that is transverse to longitudinal axis 206. In one embodiment central axis 252 is orthogonally oriented to longitudinal axis 206. The transverse and orthogonal orientations can minimize the intrusiveness into adjacent tissue when accessing stabilization member 200 to adjust the length thereof in subsequent procedures.

In another embodiment, a ratcheting type arrangement is provided such as shown in FIG. 6A. In this alternate embodiment, the stabilization member 200′ is identical to to stabilization member 200 unless otherwise noted. Stabilization member 200′ includes an adjustment mechanism 204′ with an adjustment member 230′ having drive structure 238′ about its periphery. Drive structure 238′ is in the form of ratchet teeth in the illustrated embodiment. Adjustment mechanism 204′ also includes a drive member 250′ that is oriented to extend along adjustment member 230′ in housing 240′. Drive member 250′ provides a pinion that includes teeth extending around the periphery thereof that engage drive structure 238′ in interdigitating relation. Rotation of drive member 250′ about its central axis 252′, as indicate by arrow 253′, causes adjustment member 230′ to rotate axially about longitudinal axis 206 and lengthen or shorten stabilization member 200′ depending on the direction of rotation. A locking arrangement can be provided to maintain the relative rotational positions of adjustment member 230′ and drive member 250′. In this embodiment, central axis 252′ is oriented parallel to longitudinal axis 206.

Various arrangements for engaging drive member 250, 250′ are contemplated. The drive members 250, 250′ can be provided with a head recessed to receive and engage a driver instrument, or with an external configuration around which the driver instrument is positioned. In still other embodiments, driver members 250, 250′ are rotated via magnetic or electric signals or forces from a source external to the patient or implanted with the stabilization member.

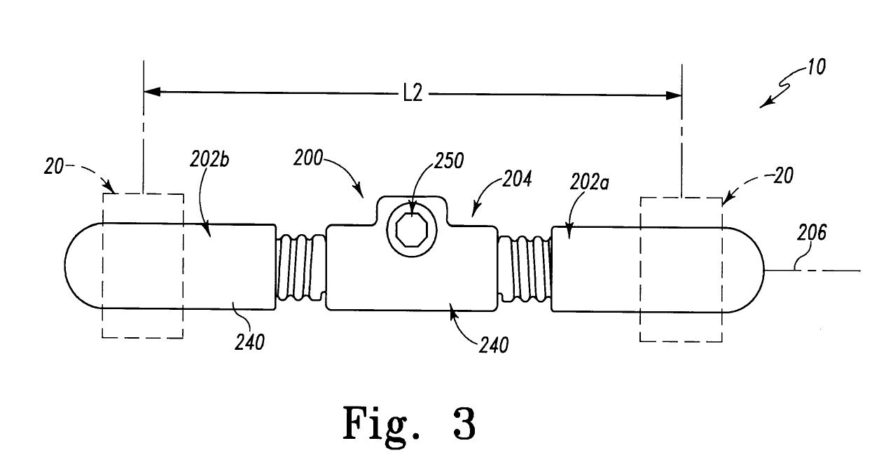

One example of using stabilization members 200, 200′ in a spinal stabilization procedure will be discussed. In FIG. 2 stabilization member 200 includes a length L1 between anchor members 20, and end portions 202a, 202b are engaged to anchor members 20 with engaging members 26. During the surgical procedure, adjustment mechanism 204 is manipulated to move end portions 202a, 202b away from one another, increasing length L1 to length L2 as shown in FIG. 3. When end portions 202a, 202b are engaged to anchor members 20, a distraction force is applied to the vertebrae through anchor members 20 by the elongated, expanded stabilization member 20.

In another example of using stabilization members 200, 200′ in a spinal stabilization procedure, stabilization member 200 includes a length L1 between anchor members 20, and end portions 202a, 202b are engaged to anchor members 20 with engaging members 26. Sometime after the surgical procedure, the length of stabilization member 200 along longitudinal axis requires post-operative adjustment to accommodate growth of the patient, to provide a different stabilization effect, or for some other reason. Adjustment mechanism 204 is accessed in a second procedure and manipulated to move end portions 202a, 202b away from one another, increasing length L1 to length L2 as shown in FIG. 3. Adjustments of the length of stabilization member 200 can further be accomplished from the length L2 in FIG. 3 to a maximum length where the end portions 202a, 202b are separated by a maximum distance to the ends of adjustment member 230, such as shown in FIG. 4, to a minimum length where end portions 202a, 202b are positioned adjacent to sleeve portion 242, such as shown in FIG. 2. The various length adjustments can be conducted in the same surgical procedure or after lapse of a period of time in one or more post-operative follow up procedures where revision surgery is deemed advisable.

In another example of using stabilization member 200 in a spinal stabilization procedure, stabilization member 200 includes a length L1 between anchor members 20. Stabilization member 200 is positioned between anchor members 20. During the surgical procedure, either before or after placement into the patient, adjustment mechanism 204 is manipulated to move end portions 202a, 202b away from one another, increasing length L1 to length L2 as shown in FIG. 3. End portions 202a, 202b are then engaged to anchor members 20 with engaging members 26 to provide an optimal length for stabilization member 200 between anchor members 20. Adjustment mechanism 204 can then be further manipulated to move end portions 202a, 202b away from one another to apply a distraction force between the vertebrae through anchor members 20, or end portions 202a, 202b are moved toward one another to apply a compression force between the vertebrae through anchor members 20. Alternatively or additionally, post-operative length adjustment is possible as deemed advisable.

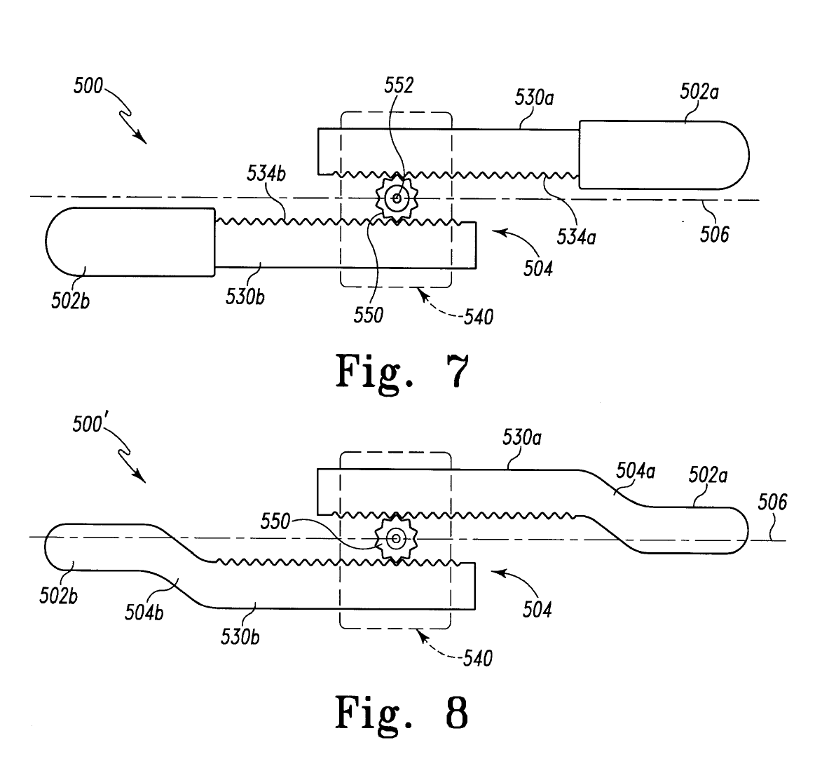

Referring now to FIG. 7, an embodiment of stabilization member 100 will be described with reference to stabilization member 500. Stabilization member 500 includes opposite end portions 502a, 502b extending along longitudinal axis 506 and an adjustment mechanism 504 axially connecting end portions 502a, 502b. End portions 502a, 502b are engageable to bony structure of the spinal column with respective ones of the anchor members 20 in the manner discussed above with respect to stabilization member 100.

End portions 502a, 502b are configured identically to one another in the illustrated embodiment, although embodiments with end portions having different configurations are also contemplated. End portions 502a, 502b can be configured with a two piece construction with a rack portion in a tubular end portion, like that discussed above for end portions 202a, 202b, or as a single, unitary piece. Adjustment mechanism 504 includes a pair of adjustment members 530a, 530b extending along longitudinal axis 506 between end portions 502a, 502b. Adjustment members 230 can include opposite engaging ends that are received in a bore of the respective end portions 502a, 502b, respectively. Alternatively, adjustment members 230a, 230b can be formed as a integral, single unit with the respective end portion 502a, 502b.

Adjustment members 530a, 530b extend through a housing 540 of adjustment mechanism 504. Housing 540 is shown in phantom lines for clarity, and can include an outer sleeve portion defining one or more longitudinal bores through which adjustment members 230a, 230b extend. Housing 540 houses a drive member 550 adjacent to and in engagement with adjustment members 530a, 530b. Drive member 550 includes a wheel like arrangement with outer teeth that interdigitate with teeth 534a, 534b along adjustment members 530a, 530b, respectively.

Drive member 550 is operable to rotate about a rotation axis 552 that is orthogonal to longitudinal axis 506 to axially translation adjustment members 530a, 530b along longitudinal axis 506 to increases or decrease the length of stabilization member 500, depending on the direction of axial rotation of adjustment member 230. End portions 502a, 502b are offset from and extend generally parallel to longitudinal axis 506. In another embodiment shown in FIG. 8, a stabilization member 500′ is shown that is generally the same as stabilization member 500. However, stabilization member 500′ includes intermediate bends 504a, 504b that connect adjustment members 530a, 530b with the respective end portions 502a, 502b so that end portions 502a, 502b are aligned with and extend along longitudinal axis 506. In another embodiment, only one bend is provided of sufficient length so that end portions 502a, 502b are aligned along a common longitudinal axis that is offset from longitudinal axis 506.

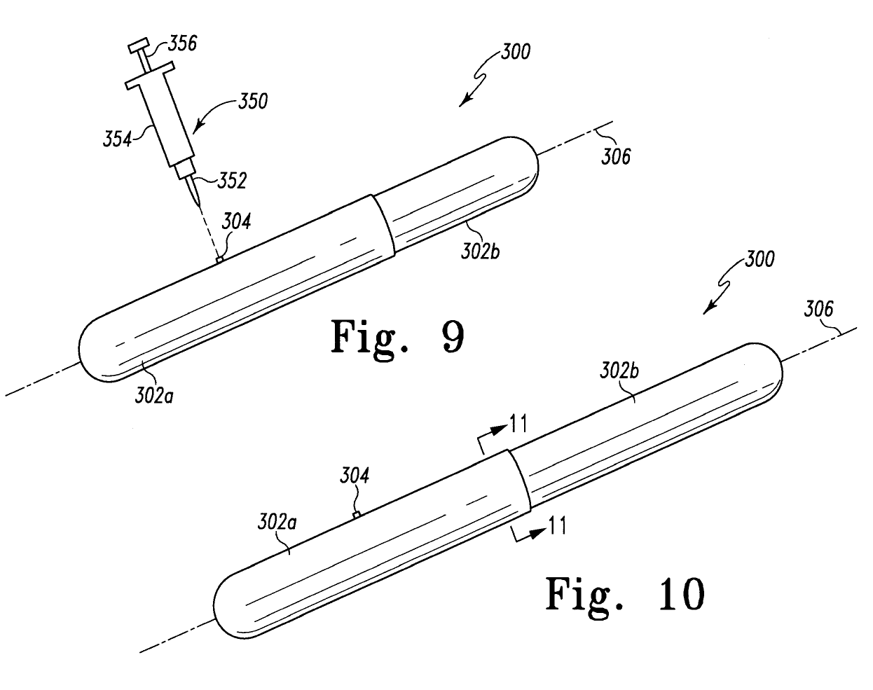

Referring now to FIGS. 9-11, there is shown another embodiment of stabilization member 100 in the form of stabilization member 300. Stabilization member 300 includes an elongated body extending along longitudinal axis 306 between a first end portion 302a and a second end portion 302b. End portions 302a, 302b overlap one another along longitudinal axis 306 in telescoping fashion. End portions 302a, 302b each include an interior bore 308 and an adjustment mechanism 304 extends from at least one of the end portions, such as end portion 302a in the illustrated embodiment. End portions 302a, 302b are movable toward and away from one another along longitudinal axis 306 to allow the length of stabilization member 300 to be adjusted. End portions 302a, 302b are engaged to bony structure of the spinal column with anchor members, such as anchor members 20 discussed above.

Adjustment mechanism 304 provides a port in communication with bores 308. Adjustment mechanism 304 includes a valve or other sealing structure in one embodiment. In another embodiment, no sealing structure is provided. A delivery device 350 includes an introducer 352 engageable to adjustment mechanism 304 to deliver an adjustment member 310 to bore 308. Adjustment member 310 is housed in chamber 354 in a flowable form, and delivered through introducer 352 by depressing a plunger 356 to force it from chamber 356. Any other suitable material delivery or dispensing system is contemplated for delivery device 350.

Adjustment member 310, as shown in FIG. 11, is delivered to bore 308 to expand the length of stabilization member 300 and move end portions 302a, 302b away from one another, as shown in FIG. 10. Additional material can be delivered to bore 308 in the same or in subsequent procedures to further adjust the length of stabilization member 300. In one embodiment, adjustment member 310 is removable to allow end portions 302a, 302b to move toward one another and decrease the length of stabilization member.

Adjustment member 310 can be any suitable bio-material deliverable to bore 308. Examples include material that readily flows or is made flowable. Examples further include material that hardens after delivery to provide a rigid stabilization member 300. Still other embodiments contemplate material that remains in fluid form after delivery. Specific examples of suitable material for adjustment member 310 include saline, PMMA bone cement, hydrogels, and polymers, to name a few.

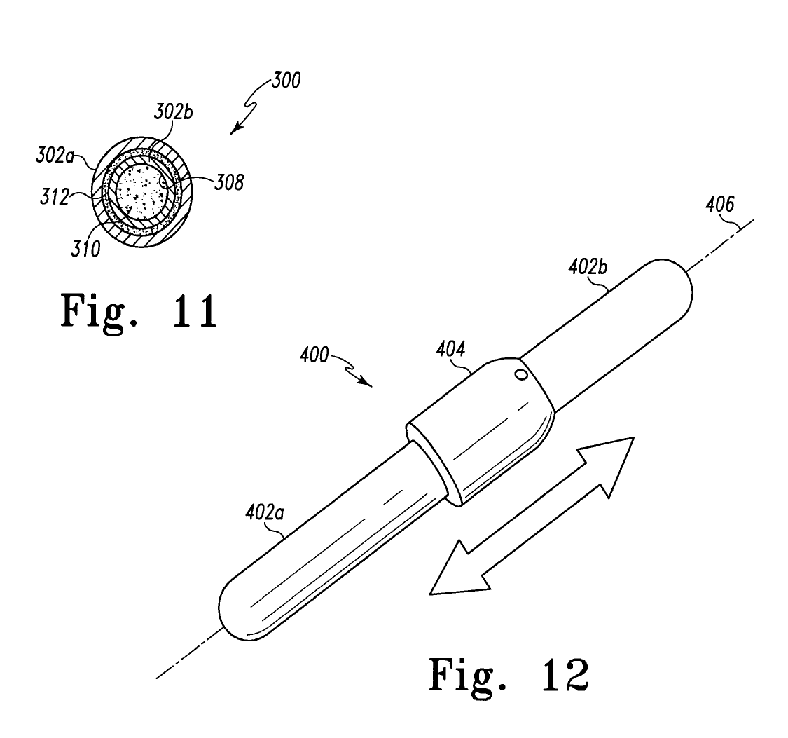

Referring now to FIG. 12, there is shown another embodiment of stabilization member 100 in the form of stabilization member 400. Stabilization member 400 includes an elongated body extending along longitudinal axis 406 between a first end portion 402a and a second end portion 402b. End portions 402a, 402b overlap one another along longitudinal axis 406 in telescoping fashion. An adjustment mechanism 404 is situated between end portions 402a, 402b, and includes an electrical mechanism that allows the length of stabilization member 400 to be adjusted along longitudinal axis 406 by electrical means. Adjustment mechanism 404 includes a servo motor in one embodiment. In another embodiment, adjustment mechanism 404 includes a piezo-electric motor.

Adjustment mechanism 404 employs electro or piezo action that articulates one of the end portions 402a, 402b to increase the overall length of stabilization member 400. In one embodiment, end portion 402a is threadingly engaged to end portion 402b, and rotation of one of the end portions 402a, 402b threadingly and axially displaces the end portions 402a, 402b relative to one another. In another embodiment, one or both of the end portions 402a, 402b is axially translated relative to the other without rotation to adjust the length of stabilization member 400. End portions 402a, 402b are engaged to bony structure of the spinal column with anchor members, such as anchor members 20 discussed above. End portions 402a, 402b are movable toward and away from one another along longitudinal axis 406 to allow the length of stabilization member 400 to be adjusted either prior to engagement to the anchors to provide optimal fit, or after engagement to the anchors to provide distraction, compression, or revision of length.

Stabilization members 300, 400 may be employed in surgical procedures such as those discussed above with respect to stabilization members 100, 200, and 200′. The surgical procedures can distract or compress vertebrae by adjusting the length of the stabilization member when engaged to anchor members, adjust the length of the stabilization member to provide an optimum fit between anchor members before engagement with the anchor member, and to provide post-operative adjustment in subsequent procedures to accommodate growth of the patient or other anatomical changes or conditions.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected.

What is claimed is:

1. A spinal stabilization system, comprising:
a stabilization member extending along a longitudinal axis between a first end portion and a second end portion, said stabilization member including an adjustment mechanism connecting said first and second end portions along said longitudinal axis; and
first and second anchor members each including a bone engaging portion to engage a bony structure and a receiving portion extending from said bone engaging portion, said receiving portions each being configured to receive a respective one of said first and second end portions, said first and second anchor members further each including an engaging member to fixedly secure said respective end portion to said receiving portion in said receptacle, wherein said adjustment mechanism is operable to move said first and second end portions toward and away from one another along said longitudinal axis to shorten and lengthen said stabilization member along said longitudinal axis with said end portions fixedly secured to said first and second anchor members.

2. The spinal stabilization system of claim 1, wherein said first and second end portions of said stabilization member are cylindrical.

3. The spinal stabilization system of claim 1, wherein said adjustment mechanism includes:
a sleeve portion including a bore extending along said longitudinal axis;
an adjustment member in said bore of said sleeve portion, said adjustment member including first and second engaging ends extending from opposite ends of said sleeve portion;
said first and second end portions each include an internal bore receiving respective ones of said first and second engaging ends therein; and
a drive member in contact with said adjustment member, said drive member being operable to rotate said adjustment member about said longitudinal axis to selectively move said first and second end portions toward and away from one another along said longitudinal axis.

4. The spinal stabilization system of claim 3, wherein said first and second end portions each define an internal thread profile along said internal bore thereof and said first and second engaging ends of said adjustment member each include an external thread profile threadingly engaged to said internal thread profile of said respective one of said first and second end portions.

5. The spinal stabilization system of claim 4, wherein said drive member includes teeth engaging a drive structure on said adjustment member.

6. The spinal stabilization system of claim 5, wherein said drive member extends along and is rotatable about a central axis that is orthogonally oriented to said longitudinal axis.

7. The spinal stabilization system of claim 4, wherein said drive member includes teeth engaging a ratchet tooth arrangement on said drive member.

8. The spinal stabilization system of claim 7, wherein said drive member extends along and is rotatable about a central axis that is oriented parallel to said longitudinal axis.

9. The spinal stabilization system of claim 1, wherein:
said first and second end portions each define an internal bore;
said first and second end portions overlap one another along said longitudinal axis in a telescoping arrangement;
said adjustment mechanism includes a port in at least one of said first and second end portions in communication with said internal bores; and
said adjustment mechanism includes an adjustment member introduced into said internal bores through said port, wherein prior to introduction of said adjustment member said stabilization member includes a first length along said longitudinal axis and after introduction of said adjustment member said stabilization member includes a second length along said longitudinal axis, said second length being greater than said first length.

10. The spinal stabilization system of claim 9, wherein said adjustment member is comprised of a material that is flowable for introduction through said port.

11. The spinal stabilization system of claim 10, wherein said material is hardenable to a second form after introduction into said internal bores.

12. The spinal stabilization system of claim 1, wherein said adjustment mechanism includes an electric motor coupled to said first and second end portions.

13. The spinal stabilization system of claim 1, wherein said adjustment mechanism includes:
a housing including a sleeve portion defining a bore extending along a longitudinal axis and a mounting portion adjacent to said sleeve portion;
an adjustment member extending through said bore between opposite first and second engaging ends, said first and second engaging ends engaged to respective ones of said first and second end portions; and
a drive member in said mounting portion engaged to said adjustment member, said drive member being operable to manipulate said adjustment member and move said first and second end portions toward and away from one another along said longitudinal axis.

14. The spinal stabilization system of claim 13, wherein said drive member is operable to rotate said adjustment member about said longitudinal axis.

15. A method for spinal stabilization, comprising:
engaging a first anchor to a first vertebra;
engaging a second anchor to a second vertebra;
engaging first and second end portions of a stabilization member to respective ones of the first and second anchors, the stabilization member including an adjustment member extending between and engaged to the first and second end portions;
manipulating the adjustment mechanism to adjust a length of the stabilization member between the first and second end portions while the stabilization member is engaged to the first and second anchors;
maintaining the stabilization member in the adjusted length; and
manipulating the adjustment mechanism to adjust the adjusted length after maintaining the adjusted length for a period of time.

16. The method of claim 15, where the first and second anchors are engaged to pedicles of the first and second vertebrae.

17. The method of claim 15, wherein the adjustment member is rotated about a longitudinal axis of the stabilization member to adjust the length of the stabilization member.

18. The method of claim 17, wherein the adjustment member is engaged to a drive member, and the drive member is rotated about its central axis to rotate the adjustment member.

19. The method of claim 18, wherein the central axis of the drive member is orthogonally oriented to the longitudinal axis of the stabilization member.

20. The method of claim 18, wherein the central axis of the drive member is oriented parallel to the longitudinal axis of the stabilization member.

21. The method of claim 15, wherein the adjustment member is introduced through a port of the stabilization member into the first and second end portions to adjust the length of the stabilization member.

22. The method of claim 21, wherein the adjustment member is comprised of a flowable material.

23. The method of claim 22, wherein the flowable material hardens after the period of time.

24. The method of claim 18, wherein the adjustment member includes an electric motor coupled to the first and second end portions of the stabilization member.

25. The method of claim 18, further comprising accessing the stabilization member in a second surgical procedure before manipulating the adjustment mechanism to adjust the adjusted length.
